(19) United States
(12) Patent Application Publication
(10) Pub. No.: US 2012/0003683 A1
(43) Pub. Date: Jan. 5, 2012

(54) CELLS FOR INDICATING THE PRESERVATION OF BIOLOGICAL SAMPLES

(75) Inventors: Alain Bonnin, Dijon (FR); Gilbert Montgaillard, Varois-et-Chaignot (FR); Patrick Ducoroy, Dijon (FR); David Vandroux, Dijon (FR)

(73) Assignees: CENTRE HOSPITALIER UNIVERSITAIRE DE DIJON, Dijon (FR); UNIVERSITIE DE BOURGOGNE, Dijon (FR); ETABLISSEMENT FRANCAIS DE SANG, Besancon (FR)

(21) Appl. No.: 13/254,912

(22) PCT Filed: Mar. 8, 2010
(86) PCT No.: PCT/EP10/52883

§ 371 Date: Sep. 6, 2011

(30) Foreign Application Priority Data
Mar. 6, 2009 (FR) 0951429

(51) Int. Cl. C12Q 1/02; C12N 5/071

(52) U.S. Cl. 435/29; 435/358

(57) ABSTRACT

Methods of characterizing the extent to which a biological sample preserved at a very low temperature has been exposed to stress using indicator cells is provided.

The present invention relates to a method for characterizing the extent to which biological samples, notably cell cultures, preserved at very low temperatures have been exposed to stress, wherein the method comprises the preservation of a sample of optionally-calibrated control (indicator) cells with said samples. Particularly, the invention relates to a method for characterizing whether a biological sample of interest, during freezing/thawing and preservation at very low temperatures, has been exposed to at least one additional stress, notably with a harmful effect for the biological sample of interest, compared to the stress caused by a cycle of freezing/thawing and preservation at very low temperatures. The invention also relates to a method for calibrating indicator cells and to the indicator cells thus calibrated.

The preservation of biological samples, notably of human origin, is a major issue in terms of public health as well as in terms of research and development of new therapies. As a result, many public and private organizations have been created in order to guarantee the optimal traceability and preservation of these samples.

Much work has shown the importance of preservation in liquid nitrogen for maintaining sample quality. Strict procedures concerning the freezing/thawing phase and the use of cryoprotectants have been defined, notably for cell therapy. However, little work has been devoted to the effect of temperature variations, notably related to the removal of samples from their normal preservation environment, for example in liquid nitrogen or in the vapor phase of liquid nitrogen, induced when new samples are added or removed, notably in preservation systems such as cryogenic storage boxes. This phenomenon can induce potentially large and iterative temperature variations that can be more or less deleterious for the sample. These temperature variations can be described as “abnormal additional” thermal stresses.

To date, there is no validated test for certifying the quality of a sample before and during its thawing.

According to a first aspect, the invention relates to a method for characterizing the extent of exposure to additional thermal stress of biological samples, in particular of cell cultures, preserved at very low temperatures, wherein the method comprises the preservation of a sample of indicator cells with said biological samples, wherein said indicator cells are calibrated to define one or more markers for distinguishing a threshold that when exceeded signifies exposure to an abnormal additional stress, notably other than that related to a normal freezing/thawing cycle.

This method can be described as “indirect” insofar as it is not carried out on the biological samples, in particular when said samples are “of interest”.

The sample of indicator cells is preserved in particular with the biological samples of interest. In particular, the biological samples of interest and the samples of indicator cells are preserved so as to undergo stresses, notably thermal stresses, in particular more or less in the same manner.

More particularly, the various samples are preserved in the same box and thus undergo identical variations in temperature. In this way, the additional stresses undergone by and measured from the indicator cells are representative of the additional stresses undergone by all of the biological samples from the same box.

Such a method can thus be used to verify whether preservation conditions are strictly followed, and in particular are in conformity with the procedures recommended and/or followed for preservation at very low temperatures. In particular, this method can be used to verify that the additional level of stress does not exceed a given threshold.

The method thus enables this verification while preserving, by leaving intact, the biological samples of interest since in the end only the indicator cells are analyzed. This is highly advantageous insofar as the samples of interest can be unique and/or precious, and insofar as it is not in particular desirable to thaw these samples to verify their quality and/or to take a portion for such a purpose.

The method according to the invention particularly characterizes so-called “additional” stress, i.e., stress other that the “normal” stress related to a single freezing/thawing cycle and to so-called “normal” preservation at very low temperatures, in particular such as defined below.

Particularly, this normal preservation at very low temperatures meets the standards and/or requirements of organizations charged with the preservation of biological samples, and in particular of cells.

These procedures can be such as those defined by CMB (Biological Resource Centers).

In the context of the present invention, “normal freezing/thawing cycle” and/or “normal preservation at very low temperatures” refers to normal freezing and thawing and/or normal preservation at very low temperatures in which, for example, once the freezing temperature or the very low temperature is reached, said temperature varies little or none at all. In particular, these variations are in conformity with the procedures recommended and/or followed for the preservation of biological samples.

In the “normal” procedure, the temperature of the sample can be lowered or returned to room temperature directly by placing the sample in an enclosure at said temperature, with an optional step in which the sample is placed beforehand in a device containing isopropanol at −80° C. for 24 hours, or 48 hours or 72 hours, or by bathing the sample in liquid nitrogen, for example for 10 minutes, before being placed at the freezing temperature.

According to a particular embodiment, the method comprises at least, or consists of, the following steps:
a) freezing at very low temperatures at least one sample of indicator cells via a normal freezing procedure,
b) freezing at very low temperatures at least one batch of biological samples of interest, in particular of cells of interest, in particular via a normal freezing procedure; this step may or may not be concomitant with step a), furthermore step b) may precede step a),
c) preservation at very low temperatures of this sample of indicator cells with at least one batch of biological samples of interest,
d) thawing of the sample of indicator cells, notably at the same time as the batch or batches of biological samples of interest, by a normal thawing procedure,
e) measurement of the marker after thawing of the indicator cells, notably during a period ranging from 5 minutes to 6 hours after thawing according to the marker measurement methods,
f) comparison of the marker measurement of step e) with a predetermined “threshold” value, notably a value that signifies exposure to additional stress, wherein in particular said exposure is such as defined in the present invention, to determine if said threshold was exceeded.

The crossing of this threshold indicates that one or more additional stresses occurred during the phases of freezing/thawing and preservation at very low temperatures.

In general, insofar as the freezing/thawing phases are well controlled and are in conformity with the normal procedure, the crossing of this threshold can indicate that additional and abnormal thermal stress occurred during preservation phase c).

In particular, steps a) to d) comprise a single normal freezing/thawing cycle.

With regard to the lowering of the temperature or to the return to room temperature, the freezing and thawing methods can be carried out according to the procedures desired, in conformity with the procedures or the regulations that are applicable or are in force and/or are the state of the art on the matter.

Several samples of indicator cells can be used, notably to verify, over time, that the cells of interest are preserved under conditions in which the additional stress is below the “threshold” value.

The predetermined threshold can be obtained in the following manner:
exposure of a sample of indicator cells to a stress related to a normal freezing/thawing cycle to which at least one additional stress is added, notably a thermal stress, then
measurement of the marker after thawing of the indicator cells, notably during a period ranging from 5 minutes to 6 hours after thawing, making it possible to define the threshold whose crossing signifies exposure to an additional stress.

The steps above can be carried out with various levels of stress and thus can provide various thresholds of stresses undergone by the biological samples of interest. For example, a first threshold can be valid in relation to a certain type of biological samples of interest, notably “fragile” samples, and a second threshold can be valid in relation to a certain type of biological samples of interest, notably “robust” samples.

Thus, the same indicator cells can be used to define different thresholds beyond which the biological samples of interest are affected according to whether they are more or less sensitive to stresses, notably thermal stresses.

Of course, these steps for determining thresholds can be carried long in advance or, on the contrary, subsequent to the thawing of the biological sample of interest, and/or independently compared to steps a) to f).

Thus, in practice, the indicator cells can be provided with tables giving correspondences between the markers, and notably their values, and the thresholds of stress or the extent of exposure to one or more additional stresses.

This can be used to determine whether the biological samples of interest were or are preserved under conditions leading to the crossing of the threshold whose crossing signifies exposure to an additional stress, without obligation to proceed to the threshold determination steps defined above.

Of course, the additional stresses can be such as defined in the present invention.

The indicator cells advantageously can be Chinese hamster ovary (CHO) cells, and more particularly CHO cells cultured in suspension (CHO-S).

The threshold, notably for affirming the occurrence of an additional stress that is abnormal to the normal freezing/thawing and preservation procedure, is preferentially identified by reference to a standard protocol of exposure to an additional stress, in particular thermal stress. This standard protocol notably can be defined by a predetermined cycle of increase and then of decrease in temperature, for example the exposure of the frozen sample to very low temperatures for two periods of 5 minutes at 20° C. followed each time by a return to the freezing temperature.

In particular, the indicator cells are placed in situations of thermal stress, i.e., exposed to temperatures higher than the preservation temperature, without the cells necessarily being thawed.

In general in these protocols the conditions, in particular sample size, are such that the sample is not thawed.

The freezing temperature can be less than or equal to −80° C., in particular less than or equal to −100° C., in particular less than or equal to −120° C., even less than or equal to −140° C., or can be −196° C.

According to another aspect, the invention relates to a method for calibrating a sample of indicator cells to define a marker for distinguishing a threshold that when exceeded signifies exposure of said indicator cells to an abnormal additional stress, notably as identified above. Said calibration method comprises the following steps:
placing the cells in culture,
incubation or culturing of the cells,
determination of the marker before freezing, for example the proportion of a population of indicator cells defined by a certain size/structure ratio, wherein the ratio between two populations of indicator cells is defined by a certain size/structure ratio and/or the level of expression of at least one molecular species.

This method of calibration can thus be used to obtain samples of indicator cells that provide at least one usable marker in a reproducible manner.

According to another aspect, the invention relates to the use of a line of CHO cells, in particular of CHO cells cultured in suspension (CHO-S), as a sample of indicator cells for characterizing the extent of exposure to an abnormal additional stress of biological samples of interest, in particular of cell cultures, preserved at very low temperatures.

In the context of the present invention, “very low temperatures” refers to temperatures less than or equal to −80° C., in particular less than or equal to −100° C., in particular less than or equal to −120° C., even less than or equal to −140° C., or a temperature of −196° C.

According to another of its aspects, the invention also relates to a line of CHO cells, notably of CHO cells cultured in suspension (CHO-S), calibrated for their use as a sample of indicator cells.

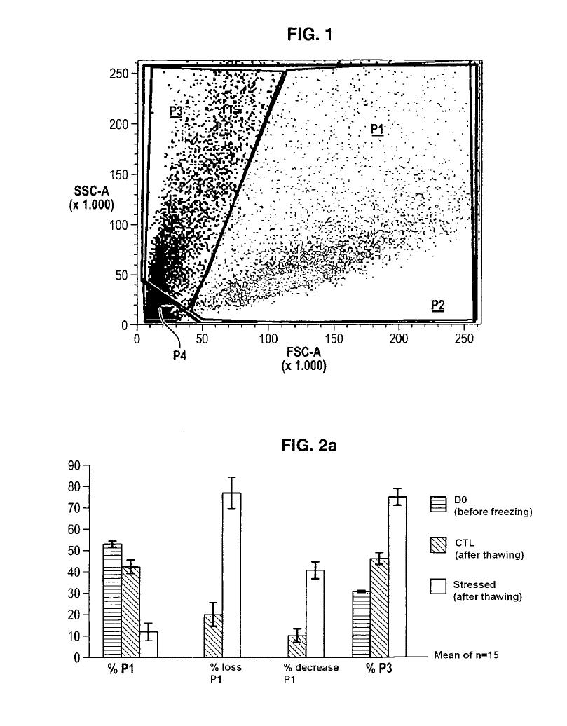

FIG. 1 represents the distribution of the various populations of CHO-S cells calibrated according to their size/structure ratio.

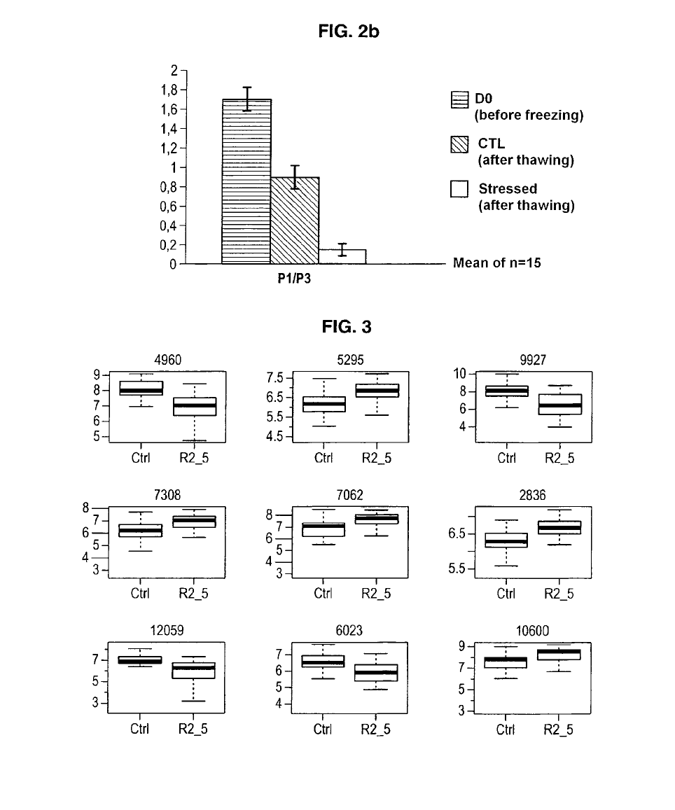

FIGS. 2a and 2b represent the distribution of two populations of calibrated CHO-S cells (populations P1 and P3) before (D0) and after freezing-thawing according to whether they undergo an additional thermal stress (stressed) or not (CTL).

FIG. 3 presents the level of expression of various peptide markers according to whether the calibrated CHO-S cells undergo an additional thermal stress (R2_{—}5) or not (Ctrl).

The indicator cells notably can be CHO cells, and in particular CHO cells cultured in suspension (CHO-S), SFM Adapted, for example those with product number 11619-012 sold by Gibco.

These cells notably have the advantage of being able to be cultured in serum-free media (SFM). Usable media include Ex-Cell 302 (BASF SE), SFC CHO Express Media (PromoCell), CHO-S SFM II (Invitrogen) or Power CHO (Lonza).

According to a particular embodiment, the marker is a change in percentage, in particular a percentage of loss, of a population, notably P1, of CHO cells before freezing and after thawing of the same sample of indicator cells.

In particular, the CHO cells are calibrated for their use as a sample of indicator cells.

Population P1 notably can be defined as being a percentage of population P2, i.e., the total number of events minus the debris (P4), and determined by a size/structure ratio of the indicator cells measured by flow cytometry.

According to a particular embodiment of the invention, with CHO-S cells as indicators, population P1 can be defined as being that whose size/structure ratio is between 0<P1 SSC<250 and 50<P1 FSC<250.

According to a more particular embodiment of the invention, with CHO cells as indicator cells, population P1 can be defined as being that whose size/structure ratio is between 0<P1 SSC<100 and 50<P1 FSC<250. In particular, population P1 is cells.

Schematically, size (side scatter, or SSC) corresponds to the scatter captured in the phase of the passage of the event, notably of the cell, in front of the laser beam, and the structure (forward scatter, or FSC) corresponds to the scatter captured at 90° during the passage of the event, in particular of the cell, in front of the laser.

In the case of CHO cells calibrated and used as indicator cells according to the present invention, the threshold characteristic of an additional stress can be induced by predetermined cycles, notably two cycles each comprising an increase to 20° C. followed by a period of 5 minutes at 20° C. and then a return to a predetermined temperature, for example, the exposure of the frozen sample at very low temperatures for two periods of 5 minutes at 20° C. followed each time by a return to the freezing temperature.

In the context of the present invention, “an increase to 20° C. followed by a period of 5 minutes at 20° C. and then a return to a predetermined temperature” means that the frozen sample is removed from the enclosure in which it is preserved at very low temperatures, that it is then placed for 5 minutes at 20° C., and that it is then returned to the enclosure in which it is preserved at very low temperatures.

In other words, the sample is taken from very low temperatures, then placed at room temperature for 5 minutes, and then returned to very low temperatures.

In particular, the characteristic threshold can be a percentage of loss of population P1 equal to or greater than 35%, in particular equal to or greater than 45%, in particular equal to or greater than 55%, even equal to or greater than 65%.

The characteristic threshold can also be a percentage of loss of population P1 of 35% to 95%, in particular of 55% to 90% and in particular of 65% to 85%.

Of course, the person skilled in the art will be able to determine this characteristic threshold according to the cell lines and the predetermined cycles selected, notably by following the method used in the examples of the embodiments below.

According to another embodiment, the value of the marker is the difference between a population, notably P1, of CHO cells calibrated for their use as a sample of indicator cells, before freezing and after thawing of the same sample of indicator cells.

The populations can be measured by flow cytometry and expressed in percentage of events in relation to population P2, i.e., the total number of events minus the debris.

This difference can be calculated by subtraction of the percentage of a population, notably of cells, in particular P1, calculated in relation to population P2, before freezing and after thawing of the same sample of indicator cells.

In particular, this difference in population, notably P1, is measured on indicator cells having been subjected to one or more additional stresses, notably thermal stresses, and in particular comprising at least one period, and in particular two periods, wherein the samples of indicator cells frozen at very low temperatures are placed at 20° C., wherein in particular the procedure is as defined above.

For CHO cells used as indicator cells according to the present invention, the difference in population P1 before freezing and after thawing and without an additional period of stress can range from 10% to 22%, notably from 15% to 19%, and in particular can be 17%.

Preferentially, for CHO cells used as indicator cells according to the present invention, the difference in population P1 before freezing and after thawing and without additional stress can range from 5% to 20%, in particular from 7% to 17%, and can be in particular 10%.

In the case of CHO cells calibrated and used as indicator cells according to the present invention, the threshold characteristic of an exposure of two periods of 5 minutes at 20° C. of samples of indicator cells frozen at very low temperatures is a difference in population P1 before freezing and after thawing of the same sample of at least 22%, in particular at least 25%, in particular at least 27%, particularly at least 30%, even at least 40%.

It is understood that the person skilled in the art will be able to determine this characteristic threshold according to the cell lines and the predetermined cycles selected.

According to another particular embodiment of the invention, the marker is a ratio between two populations P1 and P3 of CHO cells calibrated for their use as a sample of indicator cells.

Each population P1 and P3 in particular can be determined by a size/structure ratio of the indicator cells measured by flow cytometry.

According to a particular embodiment of the invention, with CHO-S cells as indicator cells, population P1 can be defined as being that whose size/structure ratio is between 0<P1 SSC<250 and 50<P1 FSC<250, more particularly this ratio can be between 0<P1 SSC<100 and 50<P1 FSC<250, and population P3 can be defined as being that whose size/structure ratio is between 50<P3 SSC<250 and 0<P3 FSC<250, more particularly this ratio can be between 50<P3 SSC<250 and 0<P3 FSC<100. In particular, populations P1 and/or P3 are cells.

In the case of CHO cells calibrated and used as indicator cells according to the present invention, the threshold characteristic of a cycle of a predetermined increase and then decrease in temperature, for example the exposure of the sample frozen at very low temperatures for two periods of 5 minutes at 20° C. followed each time by a return to the freezing temperature, can be a P1/P3 ratio ranging from 0.1 to 0.9, in particular from 0.1 to 0.7, in particular from 0.2 to 0.8, particularly from 0.2 to 0.6, even more particularly from 0.3 to 0.7, even from 0.3 to 0.5.

Of course, the person skilled in the art will be able to determine this characteristic threshold according to the cell lines and the predetermined cycles selected, in particular by following the method used in the examples of the embodiments below.

According to another embodiment of the invention, the marker is at least one molecular species, notably detectable in mass spectrometry.

The term “molecular species” can refer to any type of molecule, notably selected from proteins, polypeptides and peptides, optionally modified, for example glycosylated and/or phosphorylated, products of metabolism or fragments of one or more of these entities. Particularly, the marker is at least one polypeptide and/or at least one peptide.

A polypeptide is defined as a chain comprising from 50 to 140 amino acids linked by peptide bonds.

A peptide is defined as a chain comprising less than 50 amino acids linked by peptide bonds.

The marker can be at least one polypeptide and/or at least one peptide, in particular obtained after purification of the polypeptides and/or the peptides using hydrophobic surface chemistry, such as with Dynabeads® RPC 18 (Invitrogen), with a molecular weight of 4960 Da, 5295 Da, 9927 Da, 7308 Da, 7062 Da, 2836 Da, 12059 Da, 6023 Da, 10600 Da, 10684 Da, 3907 Da, 10970 Da, 5270 Da, 6942 Da, 2501 Da, 4935 Da, 4806 Da, 11732 Da, 11732 Da, 5861 Da, 7208 Da, 14565 Da, 7532 Da, 4506 Da, 5088 Da, 2669 Da, 6158 Da, 7078 Da, 5481 Da or 3894 Da (Dalton).

These markers notably can be used to differentiate the “control” (indicator) cells having only been subjected to stress caused by the normal preservation procedure from the “stressed” cells having been subjected to abnormal additional stress.

The marker can also be at least one polypeptide and/or at least one peptide, obtained in particular after purification of the polypeptides and/or peptides using hydrophobic surface chemistry, selected from polypeptides and peptides with molecular weights of 4960 Da, 5295 Da, 10970 Da, 7308 Da, 7062 Da, 6023 Da, 9927 Da, 10600 Da, 12059 Da, 2836 Da, 2501 Da, 10684 Da, 4506 Da, 5088 Da, 5270 Da, 7208 Da, 4935 Da, 3907 Da, 14565 Da, 5481 Da, 4806 Da, 4098 Da, 6942 Da, 6708 Da, 2669 Da, 3686 Da, 7270 Da, 8652 Da, 9962 Da and 3557 Da, in particular in order to predict whether CHO cells, in particular CHO-S cells, have or have not been stressed, notably in terms of additional stress as defined above.

The molecular weights indicated herein were obtained under the experimental conditions described below. However, other technologies, different from those cited in the present invention, can be used. In particular, examples of such technologies include mass spectrometers with a MALDI (matrix-assisted laser desorption/ionization) or ESI (electrospray ionization) source of ionization and with various analyzers and/or detectors, in particular low resolution analyzers, such as quadrupole or quadrupole (Q) mass analyzers, 3D (IT) or linear (LIT) ion trap mass analyzers, and high resolution analyzers for measuring the exact mass of analytes, such as those using a magnetic sector coupled with an electric sector, time-of-flight (TOF), Fourier transform ion cyclotron resonance (FTICR) and orbitrap.

A certain variation is likely to appear during the use of technologies and/or equipment different than those described in the present invention. Nevertheless, the person skilled in the art are able to transpose the results described in the invention to other technologies, and to identify the signals, such as for example the mass peaks obtained by mass spectrometry, corresponding to the markers characterized by the Inventors or those likely to be used as markers.

Because of variations related to the technologies, and in particular related to the specifications of the manufacturers of spectrometry equipment which notably use data processing software which can result in peak shifts, the molecular weights indicated above must be understood to have a precision of ±2%, in particular ±0.5%, even ±0.1%.

In the context of the present invention, the term “assay” refers to a semi-quantitative measurement in the broad sense, for example the relative quantification of a compound in relation to the same compound in another sample or in relation to another compound in the same sample.

Thus, in the present invention, a comparison of profiles, for example obtained by mass spectrometry, is comparable to an “assay”. The person skilled in the art can determine the number of markers to assay in order to obtain the most sensitive and selective test possible.

Among the markers characterized by the Inventors and cited above, nine were identified as being particularly relevant. They are polypeptides and/or peptides having the following molecular weights: 4960 Da, 5295 Da, 9927 Da, 7308 Da, 7062 Da, 2836 Da, 12059 Da, 6023 Da and 10600 Da. These markers are overexpressed or underexpressed in the indicator cells compared to the stressed cells, notably in terms of additional stress as defined above (FIG. 3).

Thus, the Inventors demonstrated that for CHO cells used as indicator cells, a combination of polypeptides and peptides of mass advantageously between 1000 Da and 16000 Da is characteristic of a predetermined thermal stress, defined above as being induced by predetermined cycles, notably two cycles each comprising an increase to 20° C., then a period of 5 minutes at 20° C. and then a return to the freezing temperature.

An analysis of the variation in the quantity of these peptides in the indicator cells makes it possible to identify exposure to abnormal additional stress.

Depending on the cells used to implement the inventive method, it may be necessary to calibrate said cells. This is the case notably for CHO cells sold by Gibco.

In particular, the calibration method is used to obtain indicator cells with, before freezing, a percentage of population P1, for example measured by flow cytometry, which ranges from 50% to 60%, in relation to population P2; a P1/P3 ratio, for example measured by flow cytometry, which is greater than 1; and/or a certain proportion of specific peptides.

During calibration, the indicator cells can be cultured at a temperature from 25° C. to 40° C., in particular 37° C. The atmosphere can comprise 95% v/v moisture and 5% v/v CO₂. Incubation can range from 1 day to 10 days, in particular from 2 days to 6 days, in particular 4 days.

The size/structure ratio can be measured after and during the culturing of the cells by flow cytometry.

Freezing can be carried out according to a particular procedure, in particular a procedure consistent with the freezing protocols defined by the applicable standards in the territories concerned.

In particular, the indicator cells are placed in a culture medium, for example containing 10% cold DMSO, in the form of a inoculum of 500 μl of 4·10⁶ total cells in a cryotube. Freezing can be carried out at −80° C. in a device containing isopropanol for 24 hours, or from 48 hours to 72 hours. Preservation can then take place in liquid nitrogen at a temperature below −140° C.

The cells can be thawed by incubating the cryotube for 30 seconds at 37° C. Thawing is terminated by adding cold culture medium and then the indicator cells are transferred to a centrifuge tube for centrifugation at 1000 g for 5 minutes.

In the case of protein analysis, the markers of the masses cited above can be identified according to the following procedure:

Cells (0.5·10⁶) frozen at −196° C. in 30 μl of sucrose buffer are taken up at 20° C. in 180 μl of extraction buffer and then the totality is centrifuged. After withdrawing the supernatants, the pellets are taken up in 160 μl of Milli-Q H₂O.

A volume of 50 μl is used for the purification of a peptide profile (peptidome) and a polypeptide sub-profile (polypeptidome) using C18 magnetic beads.

After the capture and elution of the polypeptides and peptides exhibiting high affinity for the phase used, Dynabeads® RPC 18 (Invitrogen), these polypeptides and peptides are analyzed by MALDI-TOF mass spectrometry (Ultraflex TOF/TOF, Bruker Daltonics, Bremen, Germany) in linear mode after mixing with the matrix α-cyano-4-hydroxycinnamic acid (HCCA) and after calibration of the instrumentation.

In the context of this invention, a differential statistical analysis was carried out, after processing of the mass spectrometry signals (normalization, baseline correction, peak detection), using the method suggested in the limma library for R which is particularly suited to large datasets related to few individuals. Predictive analysis was carried out using the random forests method. With the goal of selecting variables, the LARS model was applied.

It is understood that the person skilled in the art will be able to adapt the general calibration method of the invention to the cells which they intend to use as indicator cells in the inventive method.

According to another of its aspects, the invention also relates to a calibrated CHO cell line obtained by the inventive calibration method for the use of same as a sample of indicator cells.

According to a particular embodiment of the invention, the calibrated cells are CHO cells, in particular CHO-S cells, with a percentage of population P1, for example measured by flow cytometry, which ranges from 50% to 60%, in relation to the total number of events or to population P2, more particularly in relation to population P2, and/or a certain proportion of specific peptides.

According to another particular embodiment of the invention, the calibrated cells are CHO cells, in particular CHO-S cells, with a percentage of population P1, which can range from 50% to 60%, whose ratio between the two populations P1/P3 defined above is greater than 1, notably from 1 to 2, notably from 1.1 to 1.9, or from 1 to 1.5, even from 1.1 to 1.4.

The use of suitable mathematical models can then make it possible to categorize the samples according to their stress.

Other characteristics of the invention are illustrated in the examples of the embodiments below.

### EXAMPLES

### Example 1

CHO-S indicator cells (CHO-S cells, SFM Adapted, Gibco, product no. 11619-012) are calibrated in the following manner.

The CHO-S indicator cells (150·10³ cells/ml) are placed in a 150 cm² flask in culture in Ex-Cell 302 (BASF SE), SFC CHO Express Media (PromoCell), CHO-S SFM II (Invitrogen) or Power CHO (Lonza) containing 4 mM L-glutamine and a mixture of 19.27 mg/l hypoxanthine and 1 mg/l thymidine. These media were specially developed for these cells and do not contain serum.

Next, these indicator cells are cultured in an incubator at 37° C. under an atmosphere of 95% moisture and 5% CO₂.

The size/structure ratio of the indicator cells is observed by flow cytometry during culturing and in particular after 4 days of culture. The indicator cells (1·10⁶ cells) are suspended in 500 μl of PBS and then preserved on ice before passage in the flow cytometer.

The indicator cells in cryotubes are frozen in a culture medium containing 10% cold DMSO in the form of a 500 μl inoculum of 4·10⁶ total cells. This freezing is carried out at −80° C. in a device containing isopropanol for at least 24 hours, or 48 hours or 72 hours. The cells are then transferred to a temperature<140° C.

The indicator cells are thawed by incubating the cryotube for 30 seconds at 37° C. Thawing is terminated by adding cold culture medium, and then the cells are transferred to a centrifuge tube for centrifugation at 1000 g for 5 minutes.

FIG. 1 presents the distribution of populations P1, P2, P3 and P4 of the indicator cells calibrated according to example 1. This figure presents the distribution of the indicator cells according to their size as a function of their structure. The values are expressed in arbitrary units.

### Example 2

Two samples of calibrated indicator cells obtained according to example 1 are frozen at a temperature<−140° C.

One sample (A) does not undergo additional thermal stress while one sample (B) is subjected to two predetermined cycles each comprising an increase to 20° C., then a period of 5 minutes at 20° C. and then a return to the freezing temperature.

The two samples are then thawed according to example 1.

FIG. 2a presents the percentage of populations P1 and P3, as well as the percentage of loss of population P1, of the various samples of indicator cells before freezing (D0) and after thawing (according to the protocol defined above). FIG. 2b presents the ratio between the two populations P1 and P3 of the various samples of indicator cells before freezing (D0) and after thawing. The percentage of populations P1 and P3 were determined in relation to population P2 for cells not having undergone stress (CTL) or having been stressed by exposure to the two cycles of additional thermal stresses (stressed).

Samples A and B before freezing have a population P1 of 53% in relation to population P2. Sample A after thawing has a population P1 of 43% in relation to population P2, which is a difference in relation to population P1 of 10%.

Sample B after thawing has a population P1 of 12% in relation to population P2, which is a difference in relation to population P1 of 41%.

The percentage of loss of population P1 before freezing and after thawing of sample A is 19%, calculated as follows: 100−[(43/53)*100], whereas for the stressed sample B this difference is 77%, calculated as follows: 100−[(12/53)*100].

This measurement method shows that sample A was only subjected to thermal stress related to the normal freezing/thawing cycle whereas sample B was subjected to additional thermal stress.

Sample A after thawing has a population P3 of 47% and that of sample B is 75%. The P1/P3 ratio for sample A of 0.9 is thus calculated as follows: 43/48, whereas the ratio for sample B of 0.15 is calculated as follows: 12/75. This measurement method shows that sample A was only subjected to thermal stress related to the normal freezing/thawing cycle whereas sample B was subjected to additional thermal stress.

### Example 3

CHO-S indicator cells (0.5·10⁶ cells) calibrated according to example 1 and frozen at a temperature of −140° C. in 30 μl of sucrose buffer are taken up at 20° C. in 180 μl of extraction buffer and then the totality is centrifuged.

After withdrawing the supernatants, the pellets are taken up in 160 μl of Milli-Q H₂O. A volume of 50 μl is used for the purification of a peptide profile (peptidome) and a polypeptide sub-profile (polypeptidome) using C18 magnetic beads. After the capture and elution of the polypeptides and peptides exhibiting high affinity for the phase used (C18), these polypeptides and peptides are analyzed by MALDI-TOF mass spectrometry (Ultraflex TOF/TOF, Bruker Daltonics, Bremen, Germany) in linear mode after mixing with the matrix α-cyano-4-hydroxycinnamic acid (HOCA).

In the context of this invention, a differential statistical analysis was carried out, after processing of the mass spectrometry signals (normalization, baseline correction, peak detection), using the method suggested in the limma library for R which is particularly suited to large datasets related to few individuals. Predictive analysis was carried out using the random forests method. With the goal of selecting variables, the LARS model was applied.

FIG. 3 presents the results of these analyses on indicator cells not having undergone thermal stress (CTRL) and on indicator cells having undergone an additional stress (R2-5) according to the procedure defined in example 2.

The marker presented is effectively overexpressed or underexpressed in the indicator cells compared to the indicator cells having undergone an additional stress.

Thus, the measurement of these markers makes it possible to know if the cells were exposed to an additional stress as defined in example 2.

1. A method for characterizing the extent of exposure to a stress of biological samples preserved at very low temperatures, comprising preserving a sample of indicator cells with said samples, wherein said indicator cells are calibrated to define a marker for distinguishing a threshold that when exceeded signifies exposure to an abnormal additional stress.

2. The method of claim 1, wherein the indicator cells are Chinese hamster ovary (CHO)-cells.

3. The method of claim 1 wherein the threshold is identified by reference to a standard protocol of exposure to an additional stress.

4. The method of claim 3, wherein the standard protocol of exposure to a predetermined additional stress corresponds to two cycles each comprising an increase to 20° C., then a period of 5 minutes at 20° C. and then a return to the freezing temperature.

5. The method of claim 1, wherein said method comprises at least the following steps:
a) freezing at very low temperatures at least one sample of indicator cells via a normal freezing procedure,
b) freezing at very low temperatures at least one batch of biological samples of interest,
c) preservation at very low temperatures of this sample of indicator cells with at least one batch of biological samples of interest,
d) thawing of the sample of indicator cells,
e) measurement of the marker after thawing of the indicator cells, during a period ranging from 5 minutes to 6 hours after thawing according to the marker measurement methods,
f) comparison of the marker measurement of step e) with a predetermined “threshold” value, that signifies exposure to an additional stress to determine if said threshold was exceeded.

6. The method of claim 1, wherein the marker is a change in the percentage of a population P1 of CHO cells, before freezing and after thawing of the same sample, wherein population P1 corresponds to a percentage of population P2 that is the total number of events minus the debris, determined by flow cytometry by a size/structure ratio of the indicator cells.

7. The method of claim 6, wherein population P1 has a size/structure ratio between 0<P1 SSC<100 and 50<P1 FSC<250.

8. The method of claim 7, wherein the difference in the percentage of population P1 of CHO cells having undergone an additional stress before freezing and after thawing of the same sample of indicator cells is at least 22%.

9. The method of claim 7, wherein the percentage of loss of population P1 of CHO cells having undergone an additional stress before freezing and after thawing of the same sample of indicator cells is at least 35%.

10. The method of claim 1, wherein the marker is at least one molecular species detected by mass spectrometry.

11. The method of claim 10, wherein the marker is at least one peptide and/or at least one polypeptide.

12. The method of claim 10, wherein the peptide or the polypeptide has a molecular weight of 2501 Da, 2669 Da, 2836 Da, 3557 3686 Da, 3894 Da, 3907 Da, 4098 Da, 4506 Da, 4806 Da, 4935 Da, 4960 Da, 5088 Da, 5270 Da, 5295 Da, 5481 Da, 5861 Da, 6023 Da, 6158 Da, 6708 Da, 6942 Da, 7062 Da, 7078 Da, 7208 Da, 7270 Da, 7308 Da, 7532 Da, 8652 Da, 9927 Da, 9962 Da, 10600 Da, 10684 Da, 10970 Da, 11732 Da, 12059 Da, or 14565 Da.

13. A method for calibrating a sample of indicator cells to define a marker for distinguishing a threshold that when exceeded signifies exposure to an additional stress of biological samples preserved at very low temperatures, comprising the steps of:
placing the indicator cells in culture,
culturing of the indicator cells,
determination of the marker before freezing by the proportion of a population of indicator cells P1 defined by a certain size/structure ratio, and/or the ratio between two populations P1/P3 defined by a certain size/structure ratio and/or the level of expression of at least one molecular species.

14. (canceled)

15. (canceled)

16. A calibrated CHO cell line for use as a sample of indicator cells, characterized by a percentage of population P1 as measured by flow cytometry, which ranges from 50% to 60%, in relation to population P2 which is the total number of events minus the debris, and/or by a certain proportion of specific peptides.

17. The method of claim 1 wherein the additional stress is thermal stress.

18. The method of claim 5 wherein the biological samples of interest are cells of interest, wherein the step of freezing of the batch of the biological samples of interest is a normal freezing procedure wherein the thawing of the sample of indicator cells is at the same time as thawing of the batch or batches of biological samples of interest, and wherein the measurement of the marker after thawing of the indicator cells is carried out within a period ranging from 5 minutes to 6 hours after thawing according to the marker measurement methods.

19. The method of claim 7, wherein the difference in the percentage of population P1 of CHO cells having undergone an additional stress before freezing and after thawing of the same sample of indicator cells is at least 30%.

20. The method of claim 7, wherein the difference in the percentage of population P1 of CHO cells having undergone an additional stress before freezing and after thawing of the same sample of indicator cells is at least 40%.

21. The method of claim 7, wherein the percentage of loss of population P1 of CHO cells having undergone an additional stress before freezing and after thawing of the same sample of indicator cells is at least 55%.

22. The method of claim 7, wherein the percentage of loss of population P1 of CHO cells having undergone an additional stress before freezing and after thawing of the same sample of indicator cells is at least 65%.

23. The method of claim 1, wherein the indicator cells are Chinese hamster ovary cells cultured in suspension.

24. The method of claim 10 wherein the peptide or the polypeptide has a molecular weight of 2836 Da, 4960 Da, 5295 Da, 6023 Da, 7062 Da, 7308 Da, 9927 Da, 10600 Da or 12059 Da.
